(19) United States
(12) Patent Application Publication
(10) Pub. No.: US 2026/0298907 A1
(43) Pub. Date: Oct. 1, 2026

(54) GOUT DIAGNOSIS APPARATUS USING NONLINEAR OPTICS

(71) Applicant: PUSAN NATIONAL UNIVERSITY INDUSTRY-UNIVERSITY COOPERATION FOUNDATION, Busan (KR)

(72) Inventors: Kwang Seuk KYHM, Busan (KR); Jean-Claude Vial, Busan (KR); Jin U KIM, Busan (KR); Seung Hwan LEE, Busan (KR)

(73) Assignee: PUSAN NATIONAL UNIVERSITY INDUSTRY-UNIVERSITY COOPERATION FOUNDATION, Busan (KR)

(21) Appl. No.: 19/531,145

(22) Filed: Feb. 5, 2026

(30) Foreign Application Priority Data
Mar. 25, 2025 (KR) 10-2025-0037909

(51) Int. Cl. G01N 33/487; G01N 21/64

(52) U.S. Cl. CPC G01N 33/487; G01N 21/6402; G01N 21/6428; G01N 2800/107

(57) ABSTRACT

A gout diagnosis apparatus using nonlinear optics, includes a slide glass on which synovial fluid extracted from an affected part of a subject is placed, a laser that irradiates light onto the synovial fluid, and a detector that detects a signal by light passing through the synovial fluid and diagnoses gout or pseudogout based on whether the signal is detected.

### CROSS-REFERENCE TO PRIOR APPLICATION

This application claims priority to Korean Patent Application No. 10-2025-0037909 (filed on Mar. 25, 2025), which is hereby incorporated by reference in its entirety.

### BACKGROUND

The present disclosure relates to a gout diagnosis apparatus using nonlinear optics.

Gout and pseudogout are both diseases that cause inflammation in the joints, but there are some important differences in their causes and symptoms. First of all, gout is a disease caused by excessive accumulation of uric acid in the body, causing inflammation and pain as uric acid crystals accumulate in the joints. Gout usually causes sudden pain, swelling, and seizures in the big toes, ankles, and knees. In order to treat the gout, medications that lower uric acid levels and nonsteroidal anti-inflammatory drugs that relieve inflammation are used. On the other hand, pseudogout is caused by the accumulation of calcium pyrophosphate dihydrate crystals in the joints. It is mainly related to aging and is common in the elderly with trauma or degenerative arthritis. It causes sudden pain, swelling, and seizures in various joints, including the knees, fingers, and wrists. In order to treat this type of pseudogout, medications such as nonsteroidal anti-inflammatory drugs or colchicine are used, and in some cases, steroid injections may be prescribed.

As mentioned above, the two diseases are caused by various factors, but the crystals that cause them are different. Although symptoms presented by subjects are similar, the treatments for the two diseases are different, so an accurate diagnosis by medical staff is required. In the related art, the medical staff clinically evaluate a subject's symptoms and perform blood tests, imaging tests, and synovial fluid tests to diagnose the subject's gout and pseudogout.

In particular, synovial fluid tests are performed when more definitive evidence is needed to diagnose gout. Synovial fluid tests are performed by examining synovial fluid extracted from a joint through a polarizing microscope to observe the shape of crystals and the polarization characteristics of birefringence that cause gout or pseudogout. For example, uric acid crystals, which are gout crystals, are thinner than calcium pyrophosphate dihydrate crystals, which are pseudogout crystals, and have a needle shape, and also have different color changes in birefringence. However, the diagnosis may not be consistent during the classification process due to factors such as a level of skill of the medical staff and a degree of growth of the crystals.

Therefore, a technology that objectively distinguishes between gout and pseudogout in synovial fluid tests is urgently needed in this technical field.
Related Art 1: Korean Patent Registration No. 10-2466497
Replated Art 2: Korean Patent Registration No. 10-2361898

### SUMMARY

The present disclosure is intended to solve the foregoing problems and an aspect of the present disclosure is to obtain a gout diagnosis apparatus using nonlinear optics that detects a signal by light passing through synovial fluid and diagnoses gout or pseudogout based on whether the signal is detected in order to objectively distinguish between gout and pseudogout in synovial fluid tests.

Technical problems to be solved in the present disclosure are not limited to the above-mentioned problems and other technical problems which are not mentioned herein will definitely be understood by those skilled in the art from the following description.

In order to achieve the foregoing objective, a gout diagnosis apparatus using nonlinear optics of the present disclosure may provide a slide glass on which synovial fluid extracted from an affected part of a subject is placed; a laser that irradiates light onto the synovial fluid; and a detector that detects a signal by light passing through the synovial fluid and diagnoses gout or pseudogout based on whether the signal is detected.

As described above, according to the present disclosure, a signal may be detected by light passing through synovial fluid to diagnose gout or pseudogout based on whether the signal is detected, thereby eliminating the need for an examiner to clinically distinguish the shape of crystals in synovial fluid through a polarizing microscope, or for the examiner to read color changes due to polarization characteristics by rotating the polarizer of the polarizing microscope each time.

In addition, the present disclosure may include a band pass filter at a tip of the detector so as to exclude an autofluorescence signal from the signal and detect only a second harmonic generation signal, thereby improving diagnostic accuracy.

The effects of the present disclosure are not limited to the above-mentioned effects, and other effects that are not mentioned herein will be clearly understood by those skilled in the art from the following detailed description and appended claims.

### BRIEF DESCRIPTION OF THE DRAWINGS

FIG. 1 is a configuration diagram of a gout diagnosis apparatus using nonlinear optics of the present disclosure.

FIG. 2 is diagrams showing (a) detection of crystals in the related art and (b) detection of crystals according to one embodiment of the present disclosure.

FIG. 3 is a graph showing a signal by light passing through synovial fluid according to one embodiment of the present disclosure.

FIG. 4 is a diagram showing a single signal passing through a band pass filter according to one embodiment of the present disclosure.

FIG. 5 is a diagram showing a detection result according to one embodiment of the present disclosure.

### DETAILED DESCRIPTION

Although the terms used herein are selected from generally known and used terms considering their functions in the present disclosure, the terms may be modified depending on intention of a person skilled in the art, practices, or the advent of new technology. Besides, in a specific case, some terms may be arbitrarily chosen by the present applicant, and in this case, the meanings of those terms will be described in corresponding parts of the present disclosure in detail. Accordingly, the terms used herein should be understood not simply by the actual terms used but by the meaning lying within and the description disclosed herein.

Unless defined otherwise, the terms used herein including technological or scientific terms have the same meaning that is generally understood by those skilled in the art to which the present disclosure pertains. The terms defined in a generally used dictionary should be understood to have meanings identical to those used in the context of the related art, and are not to be construed to have ideal or excessively formal meanings unless they are obviously defined herein. Hereinafter, an embodiment according to the present disclosure will be described in detail with reference to the accompanying drawings.

A gout diagnosis apparatus 100 using nonlinear optics of the present disclosure includes a slide glass 110 on which synovial fluid A extracted from an affected part of a subject is placed, a laser 120 that irradiates light L onto the synovial fluid A, and a detector 130 that detects a signal by light L passing through the synovial fluid A and diagnoses gout or pseudogout based on whether the signal is detected.

The synovial fluid A extracted from the affected part of the subject referred to in the present disclosure may be obtained by carrying out arthrocentesis by the examiner. The arthrocentesis is a medical procedure carried out primarily to collect fluid inside a joint or to reduce pressure within the joint. Specifically, the examiner may collect synovial fluid by locally anesthetizing the affected part, disinfecting it, and then inserting a needle into the affected part of the subject. When enough synovial fluid has collected, the examiner collects it into a test tube or syringe and removes the needle. Then, the examiner apply pressure to the affected part to prevent bleeding and disinfect it to prevent infection.

The synovial fluid A dropped on an upper surface of the slide glass 110 may evaporate moisture over time so as to be fixed in the form of a film. The laser 120 may irradiate light L, particularly toward the synovial fluid A fixed to the slide glass 110 in the form of a film, on the entire area of the slide glass 110.

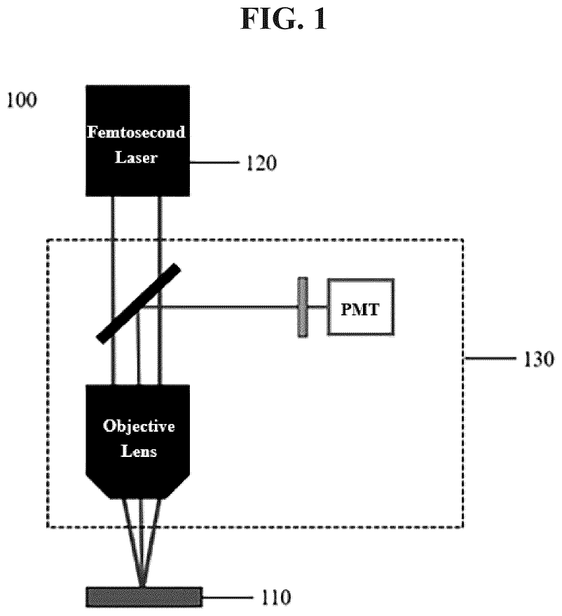

According to one embodiment of FIG. 1, the gout diagnosis apparatus 100 using nonlinear optics of the present disclosure may be an optical microscope that uses visible light to magnify an object, and combines a plurality of lenses to make the object appear larger. From the top of the apparatus 100, the laser 120, the detector 130 and the slide glass 110 may be arranged in a row in that order. The laser 120 is positioned in a direction facing the detector 130 to irradiate light L toward the slide glass 110 and the detector 130. A plurality of lenses in the detector 130 are arranged in a straight line with the laser 120 to cause phenomena such as refraction, polarization, passage, and reflection of light L. The detector 130 may detect a signal resulting from such phenomena and ultimately absorb light L.

Next, the signal may include a second harmonic generation (SHG) signal caused by monosodium urate (MSU), which is a substance related to gout.

Monosodium urate (MSU) crystals are present in gouty synovial fluid, and calcium pyrophosphate dihydrate (CPPD) crystals are present in pseudogout joint fluid. The monosodium urate (MSU) crystals are needle-shaped, and cause negative birefringence, where the birefringence of ordinary ray is greater than that of extraordinary ray, and generate a second harmonic generation signal, which is a nonlinear optical phenomenon. On the other hand, calcium pyrophosphate dihydrate (CPPD) crystals are rhombic in shape, and cause positive birefringence where the birefringence of ordinary ray is smaller than that of extraordinary ray, but do not generate a second harmonic generation signal.

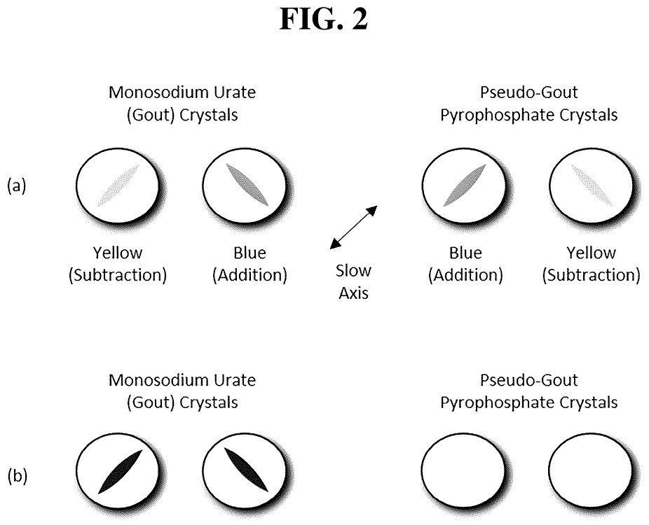

Referring to one embodiment in (a) of FIG. 2, in order to diagnose gout and pseudogout in the related art, an examiner clinically comprehensively determines the shape of the crystal, the color of the crystals, the angle of the crystals and the like through a microscope. Referring to one embodiment in (b) of FIG. 2, the detector 130 of the present disclosure may determine and diagnose that a subject has gout when a second harmonic generation signal is detected, and may determine and diagnose that the subject has pseudogout when a second harmonic generation signal is not detected. That is, the present disclosure may objectively diagnose gout or pseudogout simply by examining whether a second harmonic generation signal is generated.

Next, the laser 120 may generate femtosecond pulses at nanosecond intervals and have a near-infrared wavelength. The laser 120 may manipulate light L at a very precise and fast speed by irradiating light L in an extremely short time unit of one trillionth of a second. However, the laser 120 may generate autofluorescence, which is light emitting by itself due to external stimulation, by irradiating light L with concentrated energy in an extremely short period of time. Accordingly, the signal may further include an autofluorescence signal by monosodium urate (MSU), which is a substance related to gout, calcium pyrophosphate (CPPD), which is a substance related to pseudogout, and the synovial fluid itself.

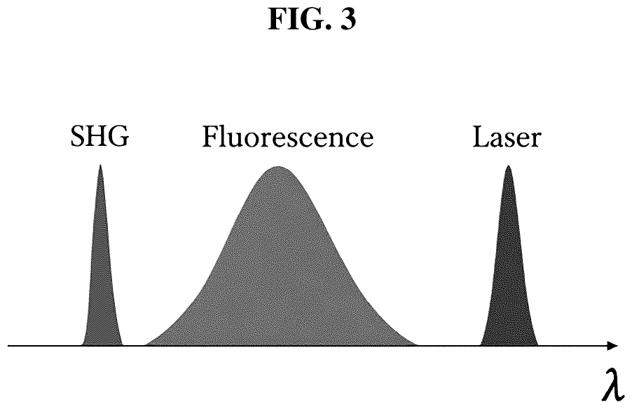

Referring to one embodiment of FIG. 3, the detector 130 may detect all signals such as a signal by light L irradiated from the laser 120, a second harmonic generation signal and an autofluorescence signal, and it is necessary to improve diagnostic accuracy by distinguishing only the second harmonic generation signal among them. The detector 130 may include a band pass filter 131 that passes a wavelength corresponding to half the wavelength of the excited light, thereby detecting only the second harmonic generation signal while excluding an autofluorescence signal generated from the synovial fluid itself.

Due to the characteristics of the signal, the autofluorescence signal is caused by the synovial fluid A, and a wavelength band emitted depending on the excited light L is not changed. On the other hand, the second harmonic generation signal is generated only at a wavelength ((½)λ) that is exactly half the wavelength λ of the excited light L.

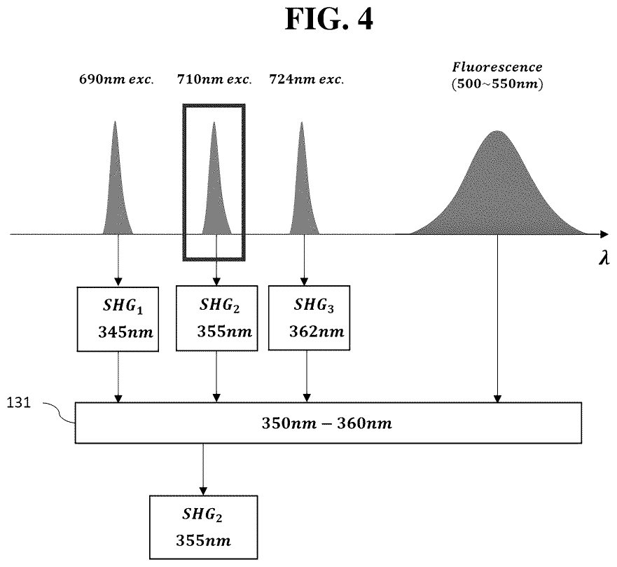

First, referring to one embodiment of FIG. 4, a wavelength λ of the excited light L may be 690 nanometers (nm), 710 nanometers (nm), and 724 nanometers (nm). In this case, the autofluorescence signal appears at 500 nanometers (nm) to 550 nanometers (nm), regardless of the wavelength λ of the excited light L. On the other hand, the second harmonic generation signal appears at a wavelength ((½)λ) that is half the wavelength λ of the excited light L, namely 345 nanometers (nm), 355 nanometers (nm), and 362 nanometers (nm). That is, the band pass filter 131 may accurately detect only the second harmonic generation signal by passing a wavelength ((½)λ) of 350 nanometers (nm) to 360 nanometers (nm) that is half the wavelength λ of the excited light L.

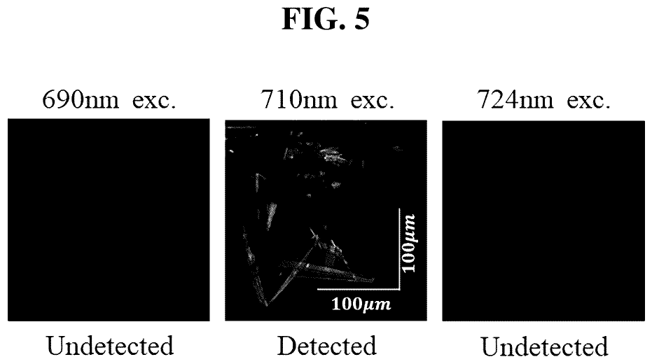

In addition, referring to one embodiment of FIG. 5, a wavelength λ of the excited light L may be 690 nanometers (nm) to 724 nanometers (nm) in a near-infrared wavelength band. In that wavelength band, both a plurality of second harmonic generation signals and a plurality of autofluorescence signals may appear. The detector 130 may include the band pass filter 131 that passes a wavelength band of 350 nanometers (nm) to 360 nanometers (nm), thereby allowing a second harmonic generation signal having a wavelength of 355 nanometers (nm) among the plurality of second harmonic generation signals to pass therethrough, and thus detecting monosodium urate crystals, which are substances related to gout.

Meanwhile, depending on the foregoing wavelength, monosodium urate crystals, which are substances related to gout, may be displayed in a blue or purple series, and the background may be displayed in black because wavelength bands other than the foregoing wavelength do not pass through the band pass filter 131. That is, the present disclosure having a near-infrared wavelength band may output monosodium urate crystals, which are substances related to gout, in a blue or purple series from the laser 120 in a red series.

As described above, though the embodiments have been described with limited embodiments and drawings, those skilled in the art may make various modifications and variations from the above description. For example, although the above-described techniques are performed in a different order from that of the above-described method, and/or the above-described components, such as a system, a structure, an apparatus, and a circuit, are coupled or combined in a different form from that of the above-described method, or replaced or substituted with other components or equivalents, proper results may be achieved.

Therefore, other implementations, other embodiments, and equivalents to the claims are within the scope of the following claims.

DESCRIPTION OF SYMBOLS

| A: | Synovial fluid |
|---|---|
| L: | Light |
| 100: | Gout Diagnosis Apparatus |
| 110: | Slide glass |
| 120: | Laser |
| 130: | Detector |
| 131: | Band pass filter |

1. A gout diagnosis apparatus using nonlinear optics, the apparatus comprising:
a slide glass on which synovial fluid extracted from an affected part of a subject is placed;
a laser that irradiates light onto the synovial fluid; and
a detector that detects a signal by light passing through the synovial fluid and diagnoses gout or pseudogout based on whether the signal is detected.

2. The apparatus of claim 1, wherein the signal comprises:
a second harmonic generation (SHG) signal by monosodium urate (MSU), which is a substance related to gout.

3. The apparatus of claim 2, wherein the detector comprises:
a band pass filter that passes a wavelength corresponding to half the wavelength of the excited light, thereby detecting only the second harmonic generation signal while excluding an autofluorescence signal generated from the synovial fluid itself.

4. The apparatus of claim 1, wherein the laser generates femtosecond pulses at nanosecond intervals, and has a near-infrared wavelength band.

5. The apparatus of claim 1, wherein the signal further comprises:
an autofluorescence signal by monosodium urate (MSU), which is a substance related to gout, calcium pyrophosphate dihydrate (CPPD), which is a substance related to pseudogout, and the synovial fluid itself.
